(19) United States
(12) Patent Application Publication
(10) Pub. No.: US 2023/0263968 A1
(43) Pub. Date: Aug. 24, 2023

(54) SYSTEM AND METHOD FOR INHALATION EXPOSURE

(71) Applicant: Data Sciences International Inc., St. Paul, MN (US)

(72) Inventors: Aaron Benjamin, Maplewood, MN (US); UV Shemesh, Minneapolis, MN (US)

(21) Appl. No.: 18/113,233

(22) Filed: Feb. 23, 2023

Related U.S. Application Data
(60) Provisional application No. 63/313,478, filed on Feb. 24, 2022.

(51) Int. Cl. A61M 15/00; A61M 16/00; A61M 16/20; A61M 16/10

(52) U.S. Cl. CPC A61M 15/0086; A61M 16/022; A61M 16/202; A61M 16/0003; A61M 16/1005

(57) ABSTRACT

Some embodiments of an inhalation exposure system can deliver a gas, such as an aerosol, through a manifold to a set of exposure chambers in a controlled manner so as to achieve improved consistency for all of the test subjects.

### CLAIM OF PRIORITY

This application claims priority under 35 U.S.C § 119 to U.S. Provisional Application Ser. No. 63/313,478 filed on Feb. 24, 2022, the entire contents of which are incorporated herein by reference.

### FIELD OF THE DISCLOSURE

The disclosure relates to an inhalation exposure system and method, and more specifically, to an inhalation exposure system that selectively and controllably delivers a gas to one or more exposure chambers.

### BACKGROUND

Some inhalants, pharmaceuticals, and other materials are tested on living subjects through exposure to an aerosol containing the material, which is then inhaled by the living subjects. In general, the testing is performed on animal subjects, e.g., mice, rats, which can be temporarily placed in an inhalation exposure chamber. The exposed subjects intake the aerosol based on a selected aerosol generation and respiration parameters, such as respiratory rate or volume dispensed over time. Simultaneously exposing multiple subjects using a single inhalation exposure system with a common aerosol source (e.g., having multiple exposure chambers) can lead to results where each subject might receive a meaningfully different quantity of the aerosol or an otherwise different amount of accumulated inhaled aerosol. For example, in an inhalation exposure system that employs a single manifold to distribute a particular concentration of aerosol to a set of exposure chambers (containing different test subjects), cessation of inhalation exposure into the manifold typically stops exposure for all subjects at once. Because each of the test subject may naturally have different respiration rates or Minute Volume (MV), each subject may have inhaled a different quantity of the aerosol during the exposure test time.

### SUMMARY

Some embodiments of an inhalation exposure system can deliver a gas, such as an aerosol, through a manifold to a set of exposure chambers in a controlled manner so as to achieve improved consistency in the amount of aerosol or gas exposure (or accumulated inhaled aerosol) for all of the test subjects. Optionally, the system can be equipped to automatically switch the gas delivery to a particular exposure chamber, responsive to a detected condition, from the aerosol passing through the manifold to a different gas source (e.g., breathable/ambient air). And such an automated change to the gas delivery to the particular exposure chamber can occur even while other exposure chambers of the inhalation exposure system continue to receive the aerosol without interruption.

A number of embodiments described herein provide an inhalation exposure system for exposing at least one subject in a chamber to an aerosol. The aerosol provides a delivery vehicle for one or more materials suspended in a gas to be delivered to and respired by the subject. In some implementations, the inhalation delivery system distributes the aerosol through a central manifold which is connected to each the exposure chamber through a respective port adapter. Optionally, the port adapter includes a reversibly collapsible valve such that, when in a first position, the aerosol is controllably delivered into a connected exposure chamber. The reversibly collapsible valve can be configured to cease delivery of the aerosol, when in a second position, and instead permit flow of a second gas (e.g., breathable air) in the connected exposure chamber.

In some embodiments, the inhalation delivery system monitors an exposure parameter (e.g., an amount of aerosol) delivered to the connected exposure chambers and, when a threshold value of the exposure parameter is reached for a particular exposure chamber, the inhalation delivery system automatically terminates the aerosol delivery to that particular exposure chamber. Optionally, the system provides a pressurized gas to an outer wall of a collapsible valve corresponding to that particular exposure chamber, and the pressurized gas causes the valve to collapse along multiple foldable wall portions (e.g., three wall portions in particular embodiments depicted herein). The wall portions adjust to a collapsed state in which the wall portions are in contact at the center of the inner lumen of the valve. Once contacted, the collapsed wall portions seal the inner lumen against gaseous flow between the central manifold and the connected exposure chamber. Additionally, in response to the collapsible valve sealing the gaseous flow from the manifold (e.g., the aerosol), the valve can contemporaneously open a flow of the pressurized gas to the exposure chamber, thereby terminating the flow of aerosol while also providing a source of respiration gas (e.g., breathable air) to the subject.

As additional description to the embodiments described below, the present disclosure describes the following embodiments. Other advantages will be apparent from the description, the drawings, and the claims. In a first example, disclosed herein is an inhalation exposure system including a set of inhalation exposure chambers in fluid communication with a gas delivery manifold, wherein each respective inhalation exposure chamber is configured to house a test subject for inhalation of a first gas. The system may include one or more sensors coupled to each respective inhalation exposure chamber to sense a characteristic of the first gas flowing from the gas delivery manifold into the respective inhalation exposure chamber. The system may also include a controller configured to simultaneously deliver the first gas from the gas delivery manifold into the set of inhalation exposure chambers and, in response to a sensed condition at one inhalation exposure chamber of the set of inhalation exposure chambers, terminate delivery of the first gas into said one inhalation exposure chamber while other inhalation exposure chambers of the set of inhalation exposure chambers continue to receive delivery of the first gas.

In some examples, responsive to termination of the delivery of the first gas, the controller provides a second gas to the one inhalation exposure chamber. The second gas can be an atmospheric gas. The system further can include at least one respective valve between the gas delivery manifold and each respective inhalation exposure chamber of the set of inhalation exposure chambers. This respective valve can include a pinch valve, a pneumatically actuated valve, or an electronically actuated valve. In some versions, the pinch valve can include an outer wall which defines an inner lumen, the inner lumen extending between an output end connected to a respective inhalation exposure chamber and an input end opposite from the inhalation exposure chamber, and wherein the outer wall can be reversibly collapsible along a plurality of foldable wall portions such that, when the outer wall can be adjusted to a collapsed configuration, the inner lumen is sealed against gaseous flow between the input end and the output end.

In a second example, disclosed herein is a method of controlling delivery of a first gas. The method may include delivering a first gas to a set of inhalation exposure chambers, and terminating delivery of the first gas into one inhalation exposure chamber of the set of inhalation exposure chambers while other inhalation exposure chambers of the set of inhalation exposure chambers continue to receive delivery of the first gas.

In some examples, the method may optionally include delivering a second gas to said one inhalation exposure chamber concurrently with terminating delivery of the first gas. The terminating can be responsive to a sensed condition at one inhalation exposure chamber of the set of inhalation exposure chambers. Additionally or alternatively, the terminating can be responsive to a signal received from user input.

In a third example, disclosed herein is an inhalation exposure system including a cylindrical valve including an outer wall which defines an inner lumen, the inner lumen extending between an output end connected to an aerosol exposure chamber and an input end opposite from the aerosol exposure chamber. The outer wall may be reversibly collapsible along a plurality of foldable wall portions such that, when the outer wall is adjusted to a collapsed configuration, the inner lumen is sealed against gaseous flow between the input end and the output end. The system may also include a housing that radially surrounds the outer wall of the cylindrical valve, the housing having a port. The system may further include a first gas source in fluid connection with the port and configured to supply a first gas to the port, and a second gas source in fluid connection with the input end of the inner lumen opposite from the aerosol exposure chamber. The second gas source may be configured to supply to the input end a second gas including an aerosol. The system may also include a sensor that communicates information indicative of an amount of the aerosol delivered to the aerosol exposure chamber. The system may further include a controller in communication with the sensor so that, in response to a determination that an exposure parameter of the aerosol delivered to the aerosol exposure chamber exceeds a threshold value, the controller supplies the first gas to adjust the outer wall of the cylindrical valve to the collapsed configuration and to flow into the aerosol exposure chamber.

In some examples, the port can be exposed toward an exterior surface of the outer wall of the cylindrical valve. The sensor can be an optical sensor. The optical sensor can be a photometer. The plurality of foldable wall portions can include three foldable wall portions. The first gas and the second gas are atmosphere. The exposure parameter can be an amount of aerosol. The exposure parameter can be an exposure time. The exposure parameter can be an accumulated dose. The exposure parameter can be an accumulated inhaled aerosol value. The first gas can be supplied to the port at a pressure in a range from 40 pounds per square inch gauge to 80 pounds per square inch gauge. The aerosol can be delivered at a rate in a range from 0.5 standard liters per minute to 1 standard liters per minute.

In a fourth example, disclosed herein is a method of controlling delivery of an aerosol. The method may include delivering an aerosol through an inner lumen of a valve and into an aerosol exposure chamber, wherein the valve has an outer wall which is reversibly collapsible to seal the inner lumen against flow of the aerosol. The method may also include, in response to an exposure parameter of the aerosol delivered to the aerosol exposure chamber exceeding a selected value, delivering a pressurized gas to an exterior of the outer wall to collapse the outer wall of the valve and seal the inner lumen against flow of the aerosol and to supply the pressurized gas to the aerosol exposure chamber.

In some examples, the exposure parameter can be an amount of aerosol. The exposure parameter can be an exposure time. The exposure parameter can be an accumulated dose. The exposure parameter can be an accumulated inhaled aerosol value. The wherein the outer wall can include three foldable wall portions, and collapsing the outer wall of the valve can include reversibly collapsing the three foldable wall portions. The method further can include generating the aerosol prior to delivering. The method further can include immobilizing a subject within the aerosol exposure chamber. The subject can be a mammal. The subject can be a murine mammal. The method further can include monitoring a respiration rate and a respiration volume of the subject. The determining the exposure parameter can include receiving information indicative of the respiration rate and a respiration volume of the subject

In a fifth example, disclosed herein is a pinch valve system including a housing including a port to receive a pressurized gas. The pinch valve system may also include a cylindrical valve including a flexible outer wall radially surrounded by the housing. The flexible outer wall can define an inner lumen and may include at least three foldable wall portions that, in response to the pressurized gas being received at the port of the housing, adjust the flexible outer wall to a collapsed position to seal the inner lumen against gaseous flow.

In a sixth example, disclosed herein is an inhalation exposure system including a valve including an inner lumen, the inner lumen extending between an output end connected to an aerosol exposure chamber and an input end opposite from the aerosol exposure chamber. The inner lumen can be adjustable such that, when the inner lumen is adjusted to a collapsed configuration, the inner lumen is sealed against gaseous flow between the input end and the output end. The system may also include a first gas source in fluid connection with the input end of the inner lumen opposite from the aerosol exposure chamber, the first gas source configured to supply to the input end a first gas including an aerosol. The system may further include a sensor that communicates information indicative of an amount of the aerosol delivered to the aerosol exposure chamber. The system may also include a controller in communication with the sensor so that, in response to a determination that an exposure parameter of the aerosol delivered to the aerosol exposure chamber exceeds a threshold value, the controller adjusts the inner lumen of the valve to the collapsed configuration and to flow the first gas into the aerosol exposure chamber.

In a seventh example, disclosed herein is a method of controlling delivery of an aerosol. The method may include delivering an aerosol through an inner lumen of a valve and into an aerosol exposure chamber, wherein the valve has an inner lumen which is reversibly adjustable to seal the inner lumen against flow of the aerosol. The method may also include, in response to a termination signal, adjusting the inner lumen of the valve to seal the inner lumen against flow of the aerosol and supplying a pressurized gas to the aerosol exposure chamber.

In some examples, the termination signal can be generated in response to the controller determining that an exposure parameter of the aerosol delivered to the aerosol exposure chamber exceeding a selected value. Additionally or alternatively, the termination signal can be generated in response to a user input.

Particular implementations of the subject matter described in this specification can be implemented so as to realize one or more of the following technical advantages.

First, some embodiments of the inhalation delivery system automatically can controllably customize inhalation exposure to each exposure chamber by determining when an exposure parameter has been met or exceeded and automatically terminating flow of the aerosol input to that particular exposure chamber. Such an improvement can advantageously increase reliability and improved consistency in the gas exposure (or accumulated inhaled aerosol) for all of the test subjects, even if the test subjects exhibit different respiration rates or Minute Volume (MV) in the respective exposure chambers.

Second, particular embodiments of the inhalation delivery system feature a controllable valve which is collapsible by applying a pressurized gas (e.g., breathable air) to the outer wall. As such, each individual exposure chamber can be readily controlled by a respective valve that is actuated with a simplified, single gas line. Also, for those embodiments in which the valve is reversibly collapsible, the system is improved by a reduced likelihood for mechanical failure.

Third, in some embodiments of the system, the gas pressure applied to the valve collapses the wall portions to simultaneously terminate aerosol flow and bleeds breathable gas to the exposure chamber for subject for continued respiration (without the aerosol). This option can increase the degree of control over the aerosol respired by each individual subject by ceasing and flushing the aerosol in a single step.

Fourth, the inhalation delivery system can controllably automate the delivery of the aerosol to each exposure chamber among a set of exposure chambers that are all connected to the same manifold. This increases throughput while maintaining the improved consistency in the gas exposure (or accumulated inhaled aerosol) for all of the test subjects.

The details of one or more embodiments are set forth in the accompanying drawings and the description below. Other features and advantages will be apparent from the description and drawings, and from the claims.

### DESCRIPTION OF DRAWINGS

FIG. 1A is a perspective view of an inhalation exposure system in accordance with some embodiments.

FIG. 1B is a cross-sectional view of the aerosol exposure system of FIG. 1A, with one exposure chamber depicted.

FIG. 2 is a cross-sectional view of an exposure chamber port adapter of the system of FIG. 1A, in accordance with some embodiments.

FIG. 3A is an exploded view of an inhalation exposure valve of the port adapter of FIG. 3.

FIG. 3B is a perspective view of an exposure nozzle of the port adapter of FIG. 3.

FIGS. 4A-4C are cross-sectional views of the collapsible valve of the port adapter of FIG. 3 shown at three configurations, in accordance with some embodiments.

FIGS. 5A-5B are cross-sectional views of an alternative embodiment of an exposure chamber port adapter for the system of FIG. 1A, in accordance with some embodiments.

FIG. 6 is a flow chart diagram of a method for delivering an aerosol to an exposure chamber.

FIG. 7 is a schematic illustration of an example computer system which can be used in conjunction with the system of FIG. 1A.

In the figures, like references indicate like elements.

### DETAILED DESCRIPTION

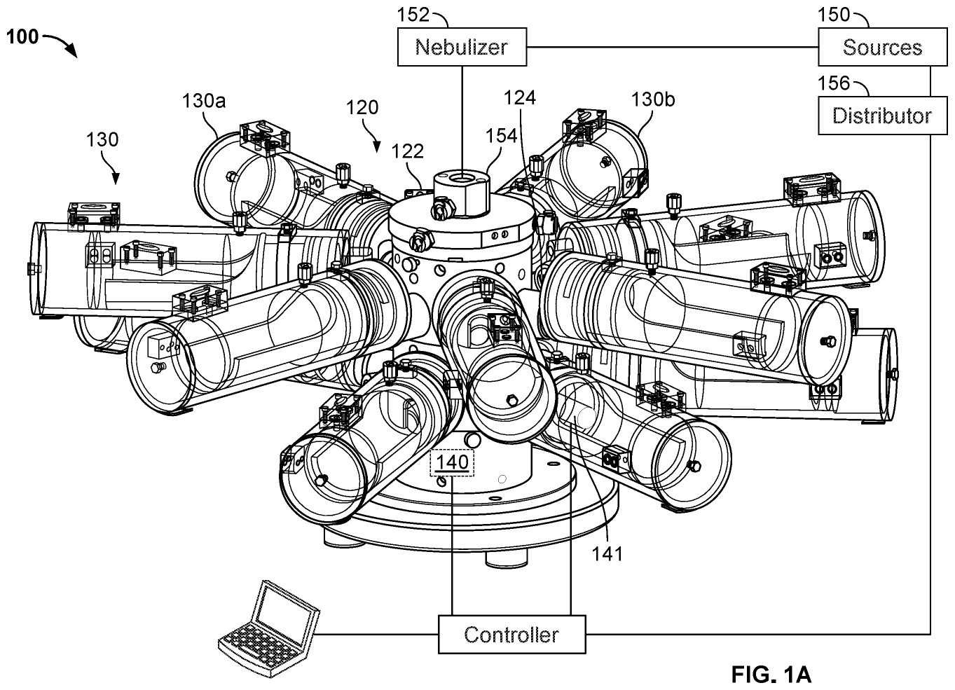

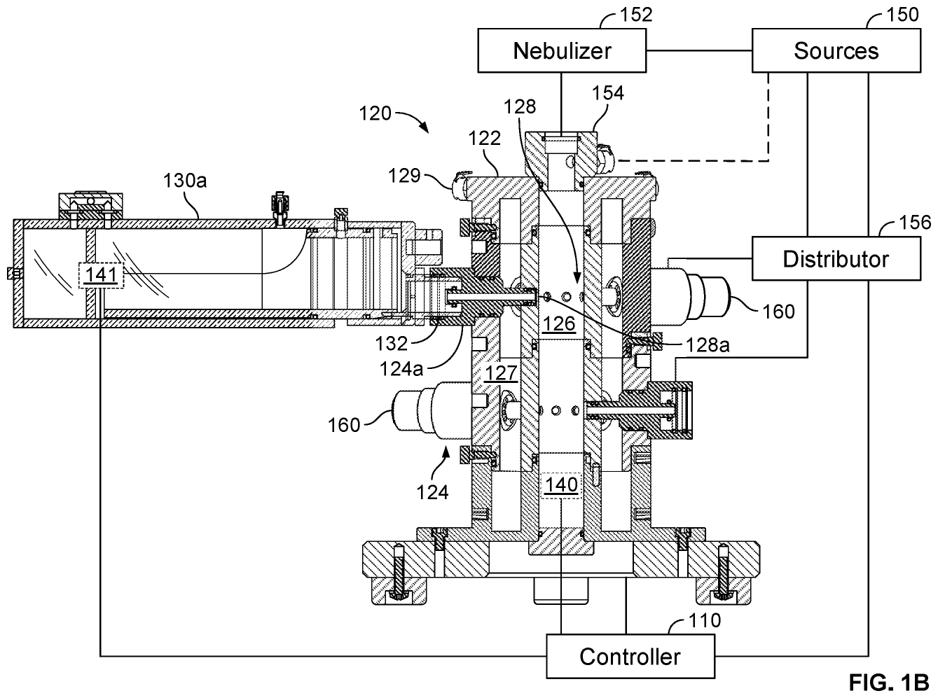

Referring to FIG. 1A, some embodiments of an inhalation exposure system 100 can include a controller 110 in communication with an exposure apparatus 120 (e.g., an exposure tower in this embodiment) so as to deliver a gas through manifold 122 to a set of aerosol exposure chambers 130 (e.g., which are configured to contain test subjects). In some implementations, the gas is an aerosol, e.g., a gas which includes suspended liquids or solids. Preferably, the gas can be delivered in a controlled manner so as to achieve improved consistency in the gas exposure (or accumulated inhaled aerosol) for all of the test subjects, even if the test subjects exhibit different respiration rates or Minute Volume (MV) in the respective exposure chambers 130. In some implementations described herein, the MV represents an estimation or calculation of the tidal volume (e.g., the amount of air that moves in or out of the lungs of a subject with each respiratory cycle) times the respiratory rate of the subject.

For example, each of the exposure chambers 130 can be configured to house an individual test subject, such as a mammal, e.g., a murine mammal (a mouse or rat), for a selected period of time, during which the inhalation exposure system 100 is configured to simultaneously delivery the gas to all of the exposure chambers 130 containing a test subject, thereby enabling the test subjects to inhale the gas.

In this embodiment, the system 100 can monitor particular characteristics of the gas delivery to each individual chamber 130. For example, the system 100 includes one or more sensors 140, 141 in connection with the controller 110 which monitor the respective characteristics of the gas (refer also to FIG. 1B). Examples of sensors 140, 141 include humidity sensors, temperature sensors, optical sensors, flow rate sensors, or scattering sensors. In response to a sensed condition, automatically switch the gas delivery to a particular chamber 130 from the aerosol passing through the manifold 122 to a different gas (e.g., breathable/ambient air), even while other exposure chambers 130 of the exposure apparatus 120 continue to receive the aerosol from the manifold 122.

Still referring to FIG. 1A, the gas is supplied by one or more gas sources 150 in fluid connection with a gas inlet 154 of the manifold 122. For example, the gas sources 150 can be pressurized canisters, or a pump. In some examples, the supplied gas can be a pure gas, or a gas mixture, e.g., atmosphere. In some implementations, the system 100 can include a nebulizer 152 in connection with the gas sources 150 which can receive the gas and introduces a material to the gas to be inhaled by the subjects. The material can include liquids, or solids. For example, the material can be liquid droplets, such as water droplets, droplets of aqueous solutions, or solid particulates, such as ash. In some implementations, the gas containing the inhalation material can be delivered the gas inlet 154 at a rate in a range from 0.1 standard liters per minute (SPLM) to 10 SPLM (e.g., from 0.5 SPLM to 1 SPLM).

The set of exposure chambers 130 includes individual exposure chambers, such as exposure chamber 130a and exposure chamber 130b. The set of exposure chambers 130 are reversibly connected to the manifold 122 by respective port assemblies 124. The port assemblies 124 can be controlled by the controller 110 to individually regulate the flow of the gas from the manifold 122 to set of exposure chambers 130. In some implementations, in which the port assemblies 124 can be actuated by gas pressure, the gas sources 150 can supply pressurized gas to a gas distributor 156 having outlets individually connected to each of the port assemblies 124. In some implementations, the gas sources 150 and the gas distributer 156 can be integrated into a single unit. In further implementations the gas sources 150 and the gas distributer 156 can be integrated into the controller 110. The controller 110 regulates pressurized gas flow from each outlet of the gas distributor 156 to individually control the gating state of the port assemblies 124. In alternative implementations, the controller 110 can have an electrical connection to electrically actuated port assemblies 124 to individually control the gating state of the port assemblies 124.

Each exposure chamber 130 can include one or more corresponding exposure sensors 141, which can receive information indicative of one or more exposure parameters of the particular subject located within a respective chamber 130, e.g., an exposure parameter value. As a first example, the exposure sensors 141 can include a plethysmograph, e.g., an instrument for recording and measuring variation in the volume of a part of the body of the subject. As a second example, the exposure sensors 141 can include an optical sensor, such as a photometer for determining an aerosol content of the gas provided to the respective exposure chamber 130.

In some implementations, the optical sensor is integrated with or otherwise attached to a corresponding port assembly 124. Alternatively or in addition to the optical sensor being attached to the port assembly 124, the exposure chamber 130 can include a flexible membrane through which a portion of the subject is placed. In one example, a subject is placed in the exposure chamber 130 such that the head (or a portion thereof, e.g., a nose) is placed through the flexible membrane. In such implementations, the gas entering the exposure chamber 130 through the port assembly 124 is substantially confined to the volume surrounding the head of the subject.

Examples of exposure parameters include a respiration rate of the subject, a respiration volume of the subject, and/or information indicative of an aerosol content in the exposure chambers 130. The controller 110 can receive the exposure parameter values, e.g., the respiration rate, respiration volume, and/or aerosol content information, and individually determine an accumulated inhaled aerosol dose for each subject in respective chambers 130. In one example, the accumulated aerosol dose for each subject can be determined by the product of respiration volume*aerosol content. In some implementations, the controller 110 can determine a time period in which the aerosol is supplied to the respective exposure chambers 130, such as the time period during which one or more port assemblies 124 are in an open state and aerosol is being provided to the respective exposure chambers 130.

The controller 110 can store, e.g., in a non-transitory media, a threshold value for each one of the exposure parameters for each subject. In some implementations, the controller 110 can store a dose threshold value for each subject in respective chambers 130. The dose threshold value can be the same value for all subjects housed within the various chambers 130, or the dose threshold value can be customized for alone or some of the subjects housed within selected chambers 130. In alternative implementations, the controller 110 can store an aerosol amount, exposure time, accumulated dose, and/or an accumulated inhaled aerosol threshold value for each subject. In some implementations, the controller 110 can receive one or more threshold values from a user before or during operation of the system 100. Additionally or alternatively, the user may stop exposure for one or more subjects arbitrarily, e.g., on demand. The user may provide input to the controller 110 to manually cease the flow of gas to one or more respective chambers 130, or the user may physically manipulate a component of the system 100 to cease the gas flow.

The controller 110 can compare the determined exposure parameter value to the respective threshold value. If the determined exposure parameter value exceeds the threshold value for a subject within an exposure chamber 130, the controller 110 can control the gating state of the respective port assemblies 124 to the closed state (which terminates the flow of aerosol to the exposure chamber 130 in which the subject is enclosed). Optionally, controlling the port assemblies 124 to the closed state contemporaneously provides the pressurized gas (e.g., breathable air) to the exposure chamber 130 instead of the previously delivered aerosol. As such, the system 100 can monitor one or more characteristics of each subject's inhalation of the aerosol in its corresponding chamber 130. Then, in response to a sensed condition, automatically switch the gas delivery to a particular chamber 130 from the aerosol passing through the manifold 122 to breathable air (absent the aerosol), even while other subjects within other exposure chambers 130 of the apparatus 120 continue to receive the aerosol from the manifold 122. In doing so, the system is configured to customize the aerosol delivery to the different subjects within the chambers, thereby permitting each subject to receive a targeted dose of the aerosol (e.g., the same dose) even when the subjects have dissimilar inhalation characteristics over time. In some implementations, a system user can select a dose threshold value for the different subjects in the chambers, and the system 100 will control the aerosol delivery to each individual chamber 130 so that the different subjects receive that selected dosage threshold value.

Referring now to FIG. 1B, a cross section through the manifold 122, exposure chamber 130a, and the respective port assembly 124a is shown. Gas from the gas sources 150 and/or nebulizer 152 can enter the manifold 122 through the gas inlet 154. The gas inlet 154 connects to an inner lumen 126 of the manifold 122. The inner lumen 126 receives the gas and includes one or more delivery ports 128 through which the port assemblies 124 connect to the inner lumen 126. In optional implementations, the nebulizer 152 is connected to the gas inlet 154 while the gas sources 150 connects downstream of the gas flow from the nebulizer 152 (shown in dashed line). The nebulizer 152 can provide the aerosol to the gas inlet 154 while the gas sources 150 can dilute and carry the aerosol.

When the port assembly 124a are in the open state, the gas is distributed through the port assemblies 124 to the connected set of exposure chambers 130. For example, port assembly 124a connects exposure chamber 130a to the inner lumen 126 through delivery port 128a.

When assembled, the set of exposure chambers 130 can define respective inner volumes which can enclose a subject. The subject can be oriented within exposure chamber 130a having the head nearest the manifold 122. The exposure chamber 130a can include a mounting adapter 132 which mates the exposure chamber 130a to the port assembly 124a. Gas from the inner lumen 126 flows through the port assembly 124a and the mounting adapter 132 into the exposure chamber 130a exposes the subject to the gas which the subject can then respire, e.g., inhale and exhale. The exposure sensors 141 receive information indicative of the respiration rate and/or volume and transmit the information to the controller 110.

The aerosol and respired gas from the subjects exit the set of exposure chambers 130 through the port assemblies 124. Outflow gas from the port assemblies 124 enters the outer lumen 127. The outer lumen 127 is separated from the inner lumen 126. The manifold 122 can include outflow ports 129 which actively or passively vent the outflow gas from the outer lumen 127. For example, in particular implementations, the outflow ports 129 can connect to a negative pressure source which may actively draw outflow gas from the outflow ports 129. In such implementations, connecting gas inlet 154 to a positive pressure source, such as gas sources 150, and outflow ports 129 to a negative pressure source can facilitate regulation of inflow to a first flow rate and varying the negative pressure facilitates maintenance of positive or negative differential pressure in the manifold 122 with respect to ambient air pressure.

Additionally or alternatively, one or more electromechanical valves connected to outflow ports 129 can vent to the surrounding atmosphere. In implementations including such valves, the valves may actuate in the event of an overpressure condition and/or to help limit fluctuations in pressure within the manifold 122.

In implementations in which one or more port assemblies 124 does not have a respective exposure chamber 130, respective plugs 160 can seal the port assemblies 124 against gaseous flow from the plugged port assemblies 124.

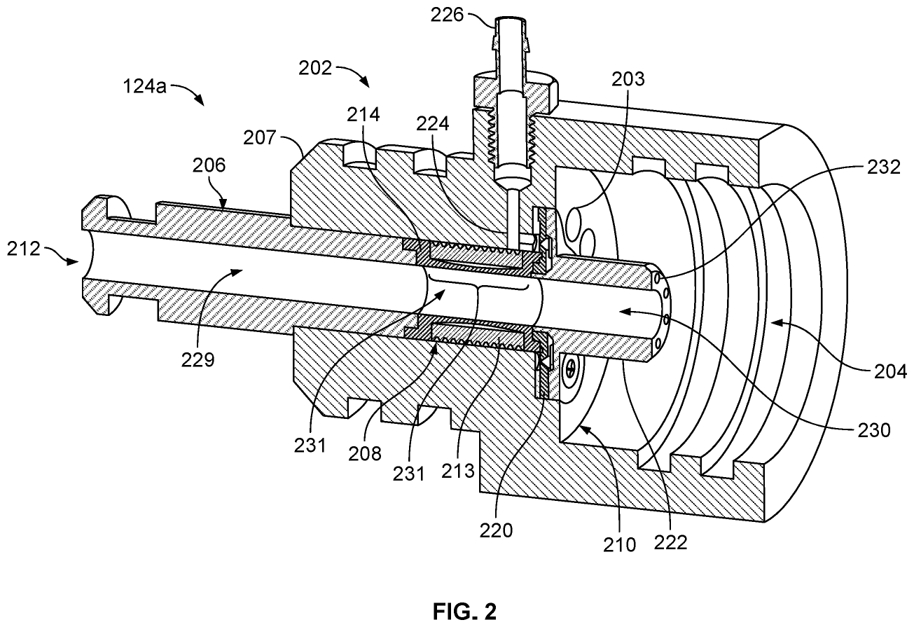

Referring now to FIG. 2, some embodiments of a port assembly 124a can include a port adapter 202, a valve 208, a flow assembly 210, and an inflow tube 206. The port assembly 124a can provide any of the port assemblies 124 of the system 100. The port adapter 202 can include a chamber receiver 204 which can provide a connection for the mounting adapter 132 to the port adapter 202. The port adapter 202 can include a manifold adapter 207 which can provide a connection for the port adapter 202 to the manifold 122. The chamber receiver 204 and/or the manifold adapter 207 can be include mating elements, e.g., threads, to connect to the respective components.

The port adapter 202, the inflow tube 206, valve 208, and flow assembly 210 include interior central channels 229, 231, and 230, respectively, which when aligned in the port adapter 202, form an inflow channel 212. The port adapter 202 can provide a support for assembling the valve 208, flow assembly 210, and inflow tube 206 such that the inflow channel 212 is aligned and permissive to gas flow. Gas from the inner lumen 126 flows through the inflow channel 212 to reach the connected exposure chamber 130a.

The valve 208 can regulate gas flow through the inflow channel 212. When in an open state the valve 208 can permit flow between exposure chamber 130a and the inner lumen 126. When in a closed state, the valve 208 can block flow between the exposure chamber 130a and the inner lumen 126. In some implementations, the valve 208 can be a pinch valve.

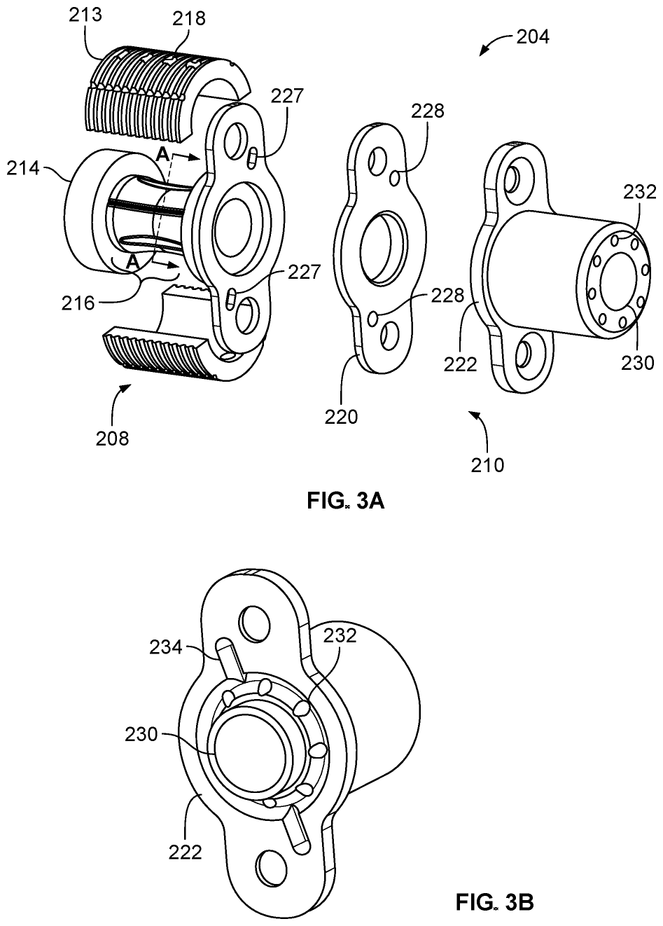

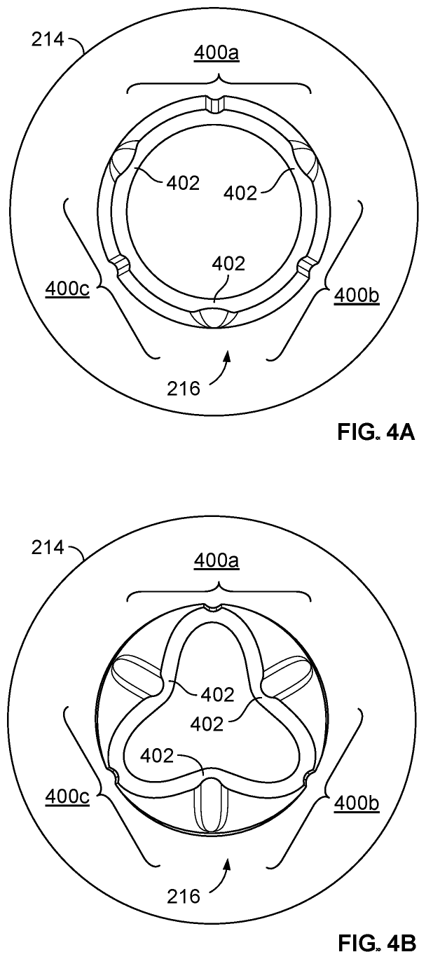

Referring to FIGS. 3A and 3B, some implementations of the flow assembly 210 include a gas director 220 and a dual flow nozzle 222. The director 220 can be arranged between the gating body 214 and the nozzle 222 and can include one or more flow passages 228 connecting opposing faces of the director 220.

The nozzle 222 can include a primary flow channel 230 connecting opposing ends, an input end and an output end, of the nozzle 222. The input end of the primary flow channel 230 is oriented away from, and the output end can be oriented toward the subject in the exposure chamber 130. The nozzle 222 can include one or more secondary flow channels 232 which can extend between opposing ends of the nozzle 222. In some implementations, the secondary flow channels 232 can be arranged radially around the primary flow channel 230. FIGS. 3A and 3B show the secondary flow channels 232 connecting the opposing ends of the nozzle 222.

The end of the nozzle 222 arranged adjacent the director 220 can include a guide ring 234. The guide ring 234 can be a shaped depression into the end of the nozzle 222 which forms a channel for gas flow and can connect one end of each of the secondary flow channels 232. The guide ring 234 can be configured to connect each of the flow passages 228 to each of the secondary flow channels 232. In some implementations, the guide ring 234 can be recessed into director 220, e.g., recessed from flow passages 228 to connect the gas to each of the secondary flow channels 232.

Referring again to FIG. 2, the valve 208 can include a rigid housing 213 surrounding a flexible gating portion 216 of a gating body 214. In some implementations, the whole valve 208 can be composed of a flexible material, e.g., rubber, or alternatively the gating portion 216 can be composed of the flexible material. The gating portion 216 can be substantially cylindrical having an outer wall configured to have a closed state and an open state. When the gating portion 216 is in the open state, the gas can flow through the valve 208 and inflow channel 212. In general, the gating portion 216 is in the open state and permissive to gas flow.

In an implementation of the valve 208, the pressurized gas can be supplied to the port adapter 202 to actuate the valve 208 and provide a second source of gas to the exposure chamber through the flow assembly 210. In some implementations, the pressurized gas can be supplied to the port adapter 202 in a range from 10 pounds per square inch gauge (PSIG) to 150 PSIG (e.g., 20 PSIG to 100 PSIG, or 40 PSIG to 80 PSIG). The port adapter 202 can include an actuation channel 224 which is configured to receive pressurized gas from an actuation inlet 226 and distribute the pressurized gas to the housing 213 and contemporaneously to flow passages 227. Some implementations of the inhalation exposure system 100 provide pressurized gas from the gas sources 150 to the actuation inlet 226 while alternative implementations provide pressurized gas from secondary gas sources (not shown).

The housing 213 can include one or more openings 218 which extends through the housing 213. The pressurized gas flows through the opening 218 and can surround the gating portion 216 within the housing 213. The gas pressure surrounding the gating portion 216 can cause the gating portion 216 to enter the closed state and cease gas flow through the inflow channel 212.

The actuation channel 224 can simultaneously provide the pressurized gas to flow passages 227. The flow passages 227 can align with flow passages 228. The flow passages 228 can provide the pressurized gas to the guide ring 234. The pressurized gas flows from the guide ring 234 through the secondary flow channels 232 and into the chamber receiver 204 and thereby into the exposure chamber 130a.

In some implementations, the gas supplied to the actuation channel 224 does not include an aerosol. When the pressurized gas is provided to the actuation inlet 226 and into the exposure chamber 130a, subject inhalation exposure ceases.

Aerosol which enters the exposure chamber 130a through the inflow channel 212 and pressurized gas which enters through the secondary flow channels 232 exits the exposure chamber 130a through outflow channels 203 in the port adapter 202. The port adapter 202 connects the chamber receiver 204 volume to the outer lumen 127 when the port adapter 202 is installed in the manifold 122. Inflow and/or pressurized gas exits the exposure chamber 130a

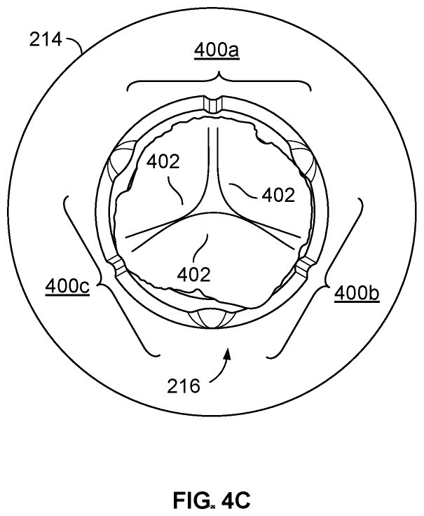

Some implementations of the gating body 214 can collapse along foldable wall portions to enter a collapsed position, e.g., the closed state. Referring now to FIGS. 4A-4C, cross sectional views through the gating portion 216 of the gating body 214 along line A of FIG. 2 are shown. In some implementations, the wall of the gating body 214 can be divided into three foldable wall portions 400, shown here as respective foldable wall portions 400a, 400b, and 400c. In some implementations, the foldable wall portions 400 can include additional fold elements which can increase the flexibility of the gating portion 216 and/or further define the motion of the foldable wall portions 400 when the gating portion 216 transitions between the opened and closed states.

The foldable wall portions 400 can be defined by flexures 402 separating each of the foldable wall portions 400. The flexures 402 are locations of increased flexibility separating the foldable wall portions 400 and can be aligned parallel to the major axis of the gating body 214. Each of the foldable wall portions 400 can include one or more flexures 402. In some implementations, the flexibility of the flexures 402 can be determined by the radial thickness of the wall of the gating portion 216, e.g., reduced wall thickness can increase the flexibility of the flexures 402.

FIG. 4A shows the open state in which there is no pressurized gas supplied to the actuation channel 224. The foldable wall portions 400 are unactuated and the gating portion 216 cross section is substantially circular. FIG. 4B shows a transition state, e.g., between the open state and the closed state, in which pressurized gas is supplied to the actuation channel 224 and surrounds the gating portion 216. The pressurized gas causes gas pressure on the outer wall of the gating portion 216 and can cause the flexures 402 to flex. When flexing, the flexures 402 can arrange toward the central axis of the gating portion 216. FIG. 4C shows the collapsed position, e.g., the closed state, of the gating portion 216. The flexures 402 contact at the central axis of the gating portion 216 to seal the inflow channel 212 against gaseous flow. When the pressurized gas is terminated, the gating portion 216 reverts to the open state.

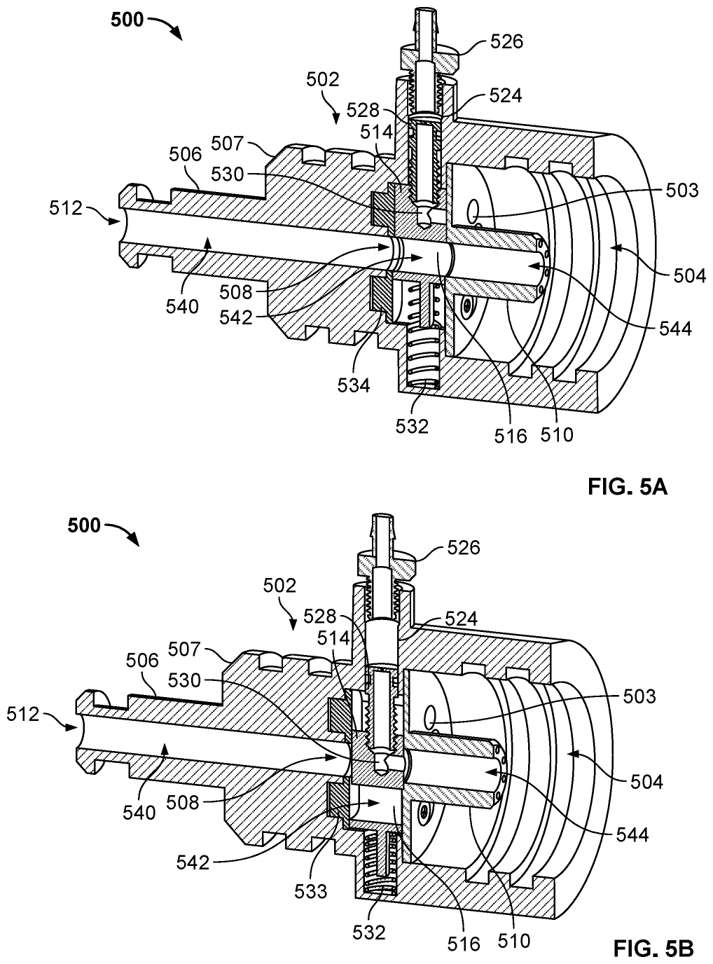

Some implementations of the port adapter 202 can include a mechanically actuated valve. FIGS. 5A and 5B show a cut-away view of an exemplary port assembly 500 having a slidable gate valve 508. The port assembly 500 can provide any of the port assemblies 124. The port assembly 500 can include a port adapter 502 having a chamber receiver 504, manifold adapter 507, and an inflow tube 506. The port adapter 502 can support a slidable gate valve 508 and a flow assembly 510. The inflow tube 506, slidable gate valve 508, and flow assembly 510 can have aligned central channels, e.g., central channels 540, 542, and 544, respectively, which can define an inflow channel 512 for gas to flow through when the slidable gate valve 508 is in an open state. The port adapter 502 can include outflow channels 503 which connect the volume of the chamber receiver 504 to the opposing face of the manifold adapter 507.

The port assembly 500 can include an actuation inlet 526 which receives pressurized gas from a connected gas source (not shown). The actuation channel 524 can receive a pressurized gas which can increase the gas pressure. A flow reducer 528 can be arranged within the actuation channel 524 and can be in contact with a gating body 514 which can be in contact on an opposing surface with a restoring element 532, e.g., a spring. Increased gas pressure in the actuation channel 524 can reposition the flow reducer 528 and gating body 514 to a closed state in which central channel 542 entirely removed from the inflow channel 512, shown in FIG. 5B, thereby preventing gas flow between central channels 540 and 544.

The gating body 514 can include a secondary channel 530 such that when the slidable gate valve 508 is in a closed state, the actuation channel 524 and the central channel 544 can be in fluid connection and can provide pressurized gas to the chamber receiver 504.

The port adapter 502 can include a sliding plate 533 arranged between the gating body 514 and the manifold adapter 507. The sliding plate 533 can be composed of a material which reduces sliding friction of the gating body 514 when transitioning between the open and closed state, such as polyethylene terephthalate (PET).

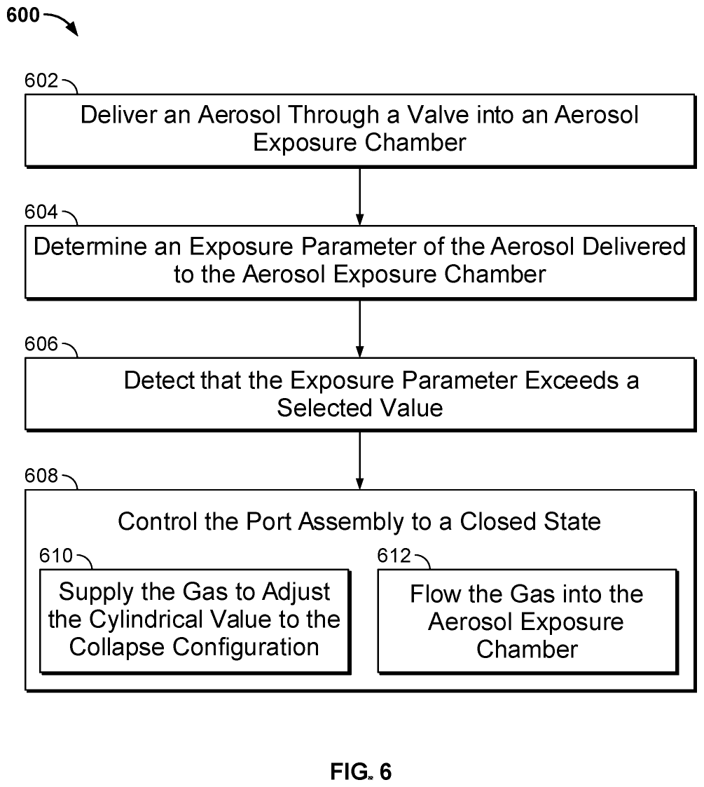

Referring now to FIG. 6, some embodiments described herein include a method 600 for controlling delivery of an aerosol to one or more of the exposure chambers 130 of the inhalation exposure system 100. Some or all of the operations in method 600 can be computer-implemented using the controller 110 or other computing device in communication with the system 100. In operation 602, the inhalation exposure system 100 delivers an aerosol to the manifold 122 from one or more gas sources 150. The manifold 122 distributes the aerosol through valves 208 of the port assemblies 124 into the connected exposure chambers 130. In some implementations, the inhalation exposure system 100 can generate the aerosol with a nebulizer 152 before being delivered to the manifold 122.

In operation 604, the controller 110 determines an exposure parameter value for a subject in a respective exposure chamber 130. The exposure parameter value can be detected via one or more sensors 141 in each chambers, and the exposure parameter value can be indicative of the subject's exposure information such as respiration rate, respiration volume, aerosol content, exposure time, accumulated does, or accumulated inhaled aerosol. In a first example, the controller 110 receives information from exposure sensors 141 in the exposure chambers 130 indicative of a respiration rate of each subject enclosed in the exposure chambers 130. In a second example, the controller 110 receives information from the exposure sensors 141 indicative of a respiration volume of the subject enclosed in the exposure chambers 130. In a third example, the controller 110 receives information from the exposure sensors 141 indicative of the aerosol delivered to each subject enclosed in the exposure chambers 130. In some implementations, the controller 110 contemporaneously receives information from exposure sensors 141 indicative of a respiration rate, a respiration volume, the amount of aerosol delivered to each subject.

In operation 606, the controller 110 detects that the received exposure parameter value for the subject exceeds a respective threshold value. For example, the controller 110 determines an accumulated aerosol dose for the subject (e.g., via the information communicated from the one or more sensors 140, 141), and then compares the aerosol dose to an aerosol dose threshold value.

As shown in operation 608, in response to detecting that the exposure parameter exceeds the respective threshold value, e.g., the aerosol dose exceeds the aerosol dose threshold value, the controller 110 terminates the delivery of the aerosol for the particular subject in that chamber 130. For example, the controller 110 can adjust the respective port assembly 124 connecting the exposure chamber 130 holding the subject to a closed state. In the embodiment depicted, for example, in FIGS. 1A-1B above, the controller 110 can be configured to control the gas distributor 156 to supply the pressurized gas (e.g., breathable air) to the inflow tube 206 which adjusts the valve 208 to the collapsed configuration, e.g., the closed state (operation 610). Also, as described above, supplying the pressurized gas to the inflow tube 206 not only causes the cessation of the aerosol delivery into that particular chamber 130, but it can contemporaneously causes that gas (e.g., breathable air) to advance into the respective exposure chamber 130 (operation 612).

In some implementations, controlling the respective port assemblies 124 can include the controller 110 sending a signal to a pneumatically actuated port assembly 124 such as the implementation described in FIG. 5. In some implementations, the port assembly 124 may be electronically actuated, e.g., such as by signals received from the controller 110 in communication with the port assembly 124.

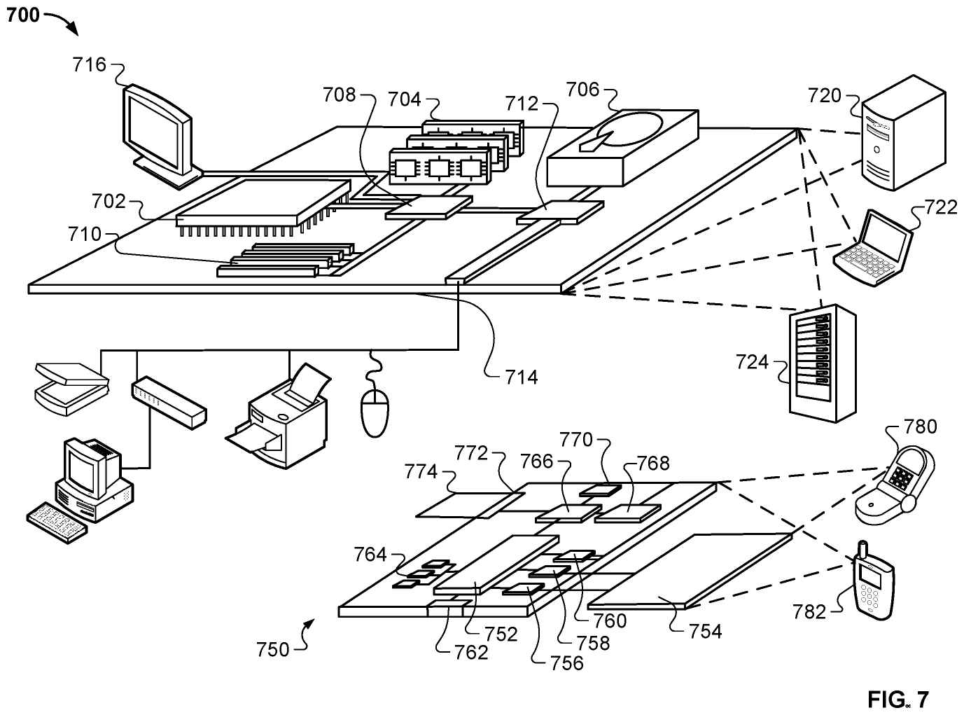

As noted previously, the systems and methods disclosed above utilize data processing apparatus to implement aspects of the inhalation exposure system described. FIG. 7 shows an example of a computing device 700 and a mobile computing device 750 that can be used as data processing apparatuses to implement the techniques described here. The computing device 700 is intended to represent various forms of digital computers, such as laptops, desktops, workstations, personal digital assistants, servers, blade servers, mainframes, and other appropriate computers. The mobile computing device 750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 766 and the storage device 706. Each of the processor 702, the memory 704, the storage device 706, the high-speed interface 708, the high-speed expansion ports 710, and the low-speed interface 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 704 stores information within the computing device 700. In some implementations, the memory 704 is a volatile memory unit or units. In some implementations, the memory 704 is a non-volatile memory unit or units. The memory 704 may also be another form of computer-readable medium, such as a magnetic or optical disk.

The storage device 706 is capable of providing mass storage for the computing device 700. In some implementations, the storage device 706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 702), perform one or more methods, such as those described above. The instructions can also be stored by one or more storage devices such as computer- or machine-readable mediums (for example, the memory 704, the storage device 706, or memory on the processor 702).

The high-speed interface 708 manages bandwidth-intensive operations for the computing device 700, while the low-speed interface 712 manages lower bandwidth-intensive operations. Such allocation of functions is an example only. In some implementations, the high-speed interface 708 is coupled to the memory 704, the display 716 (e.g., through a graphics processor or accelerator), and to the high-speed expansion ports 710, which may accept various expansion cards (not shown). In the implementation, the low-speed interface 712 is coupled to the storage device 706 and the low-speed expansion port 66. The low-speed expansion port 66, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. In addition, it may be implemented in a personal computer such as a laptop computer 722. It may also be implemented as part of a rack server system 724. Alternatively, components from the computing device 700 may be combined with other components in a mobile device (not shown), such as a mobile computing device 750. Each of such devices may contain one or more of the computing device 700 and the mobile computing device 750, and an entire system may be made up of multiple computing devices communicating with each other.

The mobile computing device 750 includes a processor 752, a memory 764, an input/output device such as a display 754, a communication interface 766, and a transceiver 768, among other components. The mobile computing device 750 may also be provided with a storage device, such as a micro-drive or other device, to provide additional storage. Each of the processor 752, the memory 764, the display 754, the communication interface 766, and the transceiver 768, are interconnected using various buses, and several of the components may be mounted on a common motherboard or in other manners as appropriate.

The processor 752 can execute instructions within the mobile computing device 750, including instructions stored in the memory 764. The processor 752 may be implemented as a chip set of chips that include separate and multiple analog and digital processors. The processor 752 may provide, for example, for coordination of the other components of the mobile computing device 750, such as control of user interfaces, applications run by the mobile computing device 750, and wireless communication by the mobile computing device 750.

The processor 752 may communicate with a user through a control interface 758 and a display interface 756 coupled to the display 754. The display 754 may be, for example, a TFT (Thin-Film-Transistor Liquid Crystal Display) display or an OLED (Organic Light Emitting Diode) display, or other appropriate display technology. The display interface 756 may comprise appropriate circuitry for driving the display 754 to present graphical and other information to a user. The control interface 758 may receive commands from a user and convert them for submission to the processor 752. In addition, an external interface 762 may provide communication with the processor 752, so as to enable near area communication of the mobile computing device 750 with other devices. The external interface 762 may provide, for example, for wired communication in some implementations, or for wireless communication in other implementations, and multiple interfaces may also be used.

The memory 764 stores information within the mobile computing device 750. The memory 764 can be implemented as one or more of a computer-readable medium or media, a volatile memory unit or units, or a non-volatile memory unit or units. An expansion memory 774 may also be provided and connected to the mobile computing device 750 through an expansion interface 772, which may include, for example, a SIMM (Single In Line Memory Module) card interface. The expansion memory 774 may provide extra storage space for the mobile computing device 750, or may also store applications or other information for the mobile computing device 750. Specifically, the expansion memory 774 may include instructions to carry out or supplement the processes described above, and may include secure information also. Thus, for example, the expansion memory 774 may be provide as a security module for the mobile computing device 750, and may be programmed with instructions that permit secure use of the mobile computing device 750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 752), perform one or more methods, such as those described above. The instructions can also be stored by one or more storage devices, such as one or more computer- or machine-readable mediums (for example, the memory 764, the expansion memory 774, or memory on the processor 752). In some implementations, the instructions can be received in a propagated signal, for example, over the transceiver 768 or the external interface 762.

The mobile computing device 750 may communicate wirelessly through the communication interface 766, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 768 using a radio-frequency. In addition, short-range communication may occur, such as using a Bluetooth, Wi-Fi, or other such transceiver (not shown). In addition, a GPS (Global Positioning System) receiver module 770 may provide additional navigation- and location-related wireless data to the mobile computing device 750, which may be used as appropriate by applications running on the mobile computing device 750.

The mobile computing device 750 may also communicate audibly using an audio codec 760, which may receive spoken information from a user and convert it to usable digital information. The audio codec 760 may likewise generate audible sound for a user, such as through a speaker, e.g., in a handset of the mobile computing device 750. Such sound may include sound from voice telephone calls, may include recorded sound (e.g., voice messages, music files, etc.) and may also include sound generated by applications operating on the mobile computing device 750.

The mobile computing device 750 may be implemented in a number of different forms, as shown in the figure. For example, it may be implemented as a cellular telephone 780. It may also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., an OLED (organic light emitting diode) display or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some embodiments, the computing system can be cloud based and/or centrally processing data. In such case anonymous input and output data can be stored for further analysis. In a cloud based and/or processing center set-up, compared to distributed processing, it can be easier to ensure data quality, and accomplish maintenance and updates to the calculation engine, compliance to data privacy regulations and/or troubleshooting.

Achieving consistent exposure or accumulated inhaled aerosol to any subject is desirable. Consistent accumulated inhaled aerosol can be achieved between subjects by stopping exposure to subjects that have reached their target exposure and continuing exposure for all other subjects. In some implementations, target accumulated inhaled aerosols can be set on per subject basis and the inhalation exposure controlled to individual exposure chambers. In some implementations, exposure durations can be achieved across subjects regardless of accumulated inhaled aerosol through the same control of inhalation exposure.

Disclosed herein is an inhalation delivery system for exposing a subject in an exposure chamber to an aerosol (e.g., a dropletized liquid) which is automatically terminable. The inhalation delivery system distributes the aerosol through a central manifold which is connected to each the exposure chamber through a port adapter. The port adapter includes a reversibly collapsible valve such that, when open, aerosol is permitted to flow into a connected exposure chamber.

The inhalation delivery system monitors the amount of aerosol delivered to the connected exposure chambers and, when a threshold is reached, closes the valve. The inhalation delivery system provides a pressurized gas to an outer wall of the collapsible valve which causes the valve to collapse along three wall portions. The wall portions collapse until contacting at the center of the inner lumen of the valve which seals against gaseous flow between the central manifold and the connected exposure chamber.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable subcombination.

What is claimed is:

1. An inhalation exposure system, comprising:
a set of inhalation exposure chambers in fluid communication with a gas delivery manifold, wherein each respective inhalation exposure chamber is configured to house a test subject for inhalation of a first gas;
one or more sensors coupled to each respective inhalation exposure chamber to sense a characteristic of the first gas flowing from the gas delivery manifold into the respective inhalation exposure chamber; and
a controller configured to simultaneously deliver the first gas from the gas delivery manifold into the set of inhalation exposure chambers and, in response to a sensed condition at one inhalation exposure chamber of the set of inhalation exposure chambers, terminate delivery of the first gas into said one inhalation exposure chamber while other inhalation exposure chambers of the set of inhalation exposure chambers continue to receive delivery of the first gas.

2. The system of claim 1, wherein the controller is configured a second gas to the one inhalation exposure chamber contemporaneously with the termination of the delivery of the first gas.

3. The system of claim 2, wherein the second gas is breathable air.

4. The system of claim 1, further comprising at least one respective valve between the gas delivery manifold and each respective inhalation exposure chamber of the set of inhalation exposure chambers.

5. The system of claim 4, wherein the least one respective valve comprises a pinch valve, a pneumatically actuated valve, or an electronically actuated valve.

6. The system of claim 5, wherein the least one respective valve comprises the pinch valves that includes an outer wall which defines an inner lumen, the inner lumen extending between an output end connected to a respective inhalation exposure chamber and an input end opposite from the inhalation exposure chamber, and wherein the outer wall is reversibly collapsible along a plurality of foldable wall portions such that, when the outer wall is adjusted to a collapsed configuration, the inner lumen is sealed against gaseous flow between the input end and the output end.

7. A method of controlling delivery of a first gas, comprising:
delivering a first gas to a set of inhalation exposure chambers; and
terminating delivery of the first gas into one inhalation exposure chamber of the set of inhalation exposure chambers while other inhalation exposure chambers of the set of inhalation exposure chambers continue to receive delivery of the first gas.

8. The method of claim 7, further comprising delivering a second gas to said one inhalation exposure chamber concurrently with terminating delivery of the first gas.

9. The method of claim 7, wherein the terminating is responsive to a sensed condition at said one inhalation exposure chamber of the set of inhalation exposure chambers.

10. The method of claim 7, wherein the terminating is responsive to a signal received from user input.

11. An inhalation exposure system, comprising:
a cylindrical valve comprising an outer wall which defines an inner lumen, the inner lumen extending between an output end connected to an aerosol exposure chamber and an input end opposite from the aerosol exposure chamber, and wherein the outer wall is reversibly collapsible along a plurality of foldable wall portions such that, when the outer wall is adjusted to a collapsed configuration, the inner lumen is sealed against gaseous flow between the input end and the output end;
a housing that radially surrounds the outer wall of the cylindrical valve, the housing having a port;
a first gas source in fluid connection with the port and configured to supply a first gas to the port;
a second gas source in fluid connection with the input end of the inner lumen opposite from the aerosol exposure chamber, the second gas source configured to supply to the input end a second gas comprising an aerosol;
a sensor that communicates information indicative of an amount of the aerosol delivered to the aerosol exposure chamber; and
a controller in communication with the sensor so that, in response to a determination that an exposure parameter of the aerosol delivered to the aerosol exposure chamber exceeds a threshold value, the controller supplies the first gas to adjust the outer wall of the cylindrical valve to the collapsed configuration and to flow into the aerosol exposure chamber.

12. The system of claim 11, wherein the port is exposed toward an exterior surface of the outer wall of the cylindrical valve.

13. The system of claim 11, wherein the sensor is an optical sensor.

14. The system of claim 13, wherein the optical sensor is a photometer.

15. The system of claim 11, wherein the plurality of foldable wall portions comprises three foldable wall portions.

16. The system of claim 11, wherein the first gas and the second gas are atmospheric gas.

17. The system of claim 11, wherein the exposure parameter is selected from a group consistent of an amount of aerosol, an exposure time, an accumulated dose, and an accumulated inhaled aerosol value.

18. The system of claim 11, wherein the first gas is supplied to the port at a pressure in a range from 40 pounds per square inch gauge to 80 pounds per square inch gauge.

19. The system of claim 11, wherein the aerosol is delivered at a rate in a range from 0.5 standard liters per minute to 1 standard liters per minute.
